(19) United States
(12) Patent Application Publication
(10) Pub. No.: US 2016/0354022 A1
(43) Pub. Date: Dec. 8, 2016

(54) METHOD OF ESTIMATING INTERNET ACTIVITY DEPENDENCE OF A HUMAN SUBJECT

(71) Applicant: National Chiao Tung University, Hsinchu City (TW)

(72) Inventors: Tzu-Chien Hsiao, Hsinchu City (TW); Hong-Ming Ji, Nantou County (TW); Dai-Ling Hsieh, Tainan City (TW)

(21) Appl. No.: 15/046,882

(22) Filed: Feb. 18, 2016

(30) Foreign Application Priority Data
Jun. 8, 2015 (TW) 104118495

(51) Int. Cl. A61B 5/16; A61B 5/00; A61B 5/113

(52) U.S. Cl. CPC A61B 5/16; A61B 5/1135; A61B 5/742; A61B 5/7278; A61B 5/7203; A61B 5/7246; A61B 5/6802

(57) ABSTRACT

In a method of estimating Internet activity dependence of a human subject, a respiration sensing unit senses respiration-caused thoracic and abdominal movements of the human subject during a first time period, and a subsequent second time period during which an emotion stimulating material played on a multimedia playing unit is being watched by the human subject so as to generate two sets of thoracic and abdominal respiratory signals corresponding respectively to the first and second time periods. A processing unit generates an estimation result associated with the Internet activity dependence based on the emotion stimulating material and the sets of thoracic and abdominal respiratory signals.

### CROSS-REFERENCE TO RELATED APPLICATION

This application claims priority of Taiwanese Application No. 104118495, filed on Jun. 8, 2015.

### FIELD

The disclosure relates to estimation of Internet activity dependence of a human subject, and more particularly to a method of estimating Internet activity dependence of a human subject.

### BACKGROUND

In recent years, it has become very convenient to acquire and use the Internet. However, with such convenience come undesirable habitual behaviors, such as excessive Internet usage, and addiction to the Internet in gaming and shopping.

An Internet addict may always feel tired and/or have unstable emotions to cause an estranged relationship with his/her family members, and to be unable to concentrate, thereby adversely affecting, e.g., school or workplace performance. In view of the above problems, a topic to explore correlation between Internet activity dependence and Internet addiction has been taken seriously.

In a typical estimation method for Internet activity dependence of an Internet user, first of all, the Internet user is required to frequently fill out specific questionnaire throughout an entire span of the period of estimation, e.g., six months. Then, the Internet activity dependence of the Internet user corresponding to the period of estimation can be estimated based on all the questionnaires filled out by the Internet user during the period of estimation. Such estimation method is not only time-consuming, but also unsuitable for adequately estimating a current Internet activity dependence of the Internet user.

### SUMMARY

Therefore, an object of the disclosure is to provide a method of estimating Internet activity dependence of a human subject that can overcome the drawbacks of the prior art.

According to the disclosure, there is provided a method of estimating Internet activity dependence of a human subject. The method is to be implemented by a system that includes a respiration sensing unit and a processing unit. The method includes the steps of:

A) by the respiration sensing unit, sensing respiration-caused thorax and abdomen movements of the human subject during a first time period, and during a subsequent second time period during which an emotional stimulation material played on a multimedia playing unit is being watched by the human subject, so as to generate a first thoracic respiratory signal and a first abdominal respiratory signal that correspond to the first time period, and a second thoracic respiratory signal and a second abdominal respiratory signal that correspond to the second time period; and

B) by the processing unit, generating an estimation result associated with the Internet activity dependence of the human subject based on a predetermined criterion associated with the emotional stimulation material, the first and second thoracic respiratory signals and the first and second abdominal respiratory signals.

### BRIEF DESCRIPTION OF THE DRAWINGS

Other features and advantages of the disclosure will become apparent in the following detailed description of the embodiments with reference to the accompanying drawings, of which:

FIG. 1 is a schematic block diagram illustrating a system that is configured for implementing the first embodiment of a method of estimating Internet activity dependence of a human subject according to the disclosure;

FIG. 2 is a flow chart illustrating the first embodiment of the disclosure;

FIG. 3 is a flow chart illustrating how a processing unit generates an estimation result based on a predetermined criterion associated with an emotional stimulation material including positive emotional content, and on respiratory signals (St₁, St₂, Sa₁, Sa₂) in the first embodiment;

FIG. 3A is an exemplary flow chart illustrating how the processing unit generates, the estimation result based on a predetermined criterion associated with the emotional stimulation material including the positive emotional content, and on first data and second data in the first embodiment;

FIG. 3B is another exemplary flow chart illustrating how the processing unit generates, the estimation result based on the predetermined criterion associated with the emotional stimulation material including the positive emotional content, and on the first and second data in the first embodiment;

FIG. 4 is a flow chart illustrating the second embodiment of a method of estimating Internet activity dependence of a human subject according to the disclosure;

FIG. 5 is a flow chart illustrating how the processing unit generates an estimation result based on a predetermined criterion associated with an emotional stimulation material including negative emotional content, and on respiratory signals (St₁, St₂, Sa₁, Sa₂) in the second embodiment;

FIG. 5A is an exemplary flow chart illustrating how the processing unit generates, the estimation result based on the predetermined criterion associated with the emotional stimulation material including the negative emotional content, and on first data and second data in the second embodiment; and

FIG. 5B is another exemplary flow chart illustrating how the processing unit generates the estimation result based on the predetermined criterion associated with the emotional stimulation material including the negative emotional content, and on the first and second data in the second embodiment.

### DETAILED DESCRIPTION

Before the disclosure is described in greater detail, it should be noted that like elements are denoted by the same reference numerals throughout the disclosure.

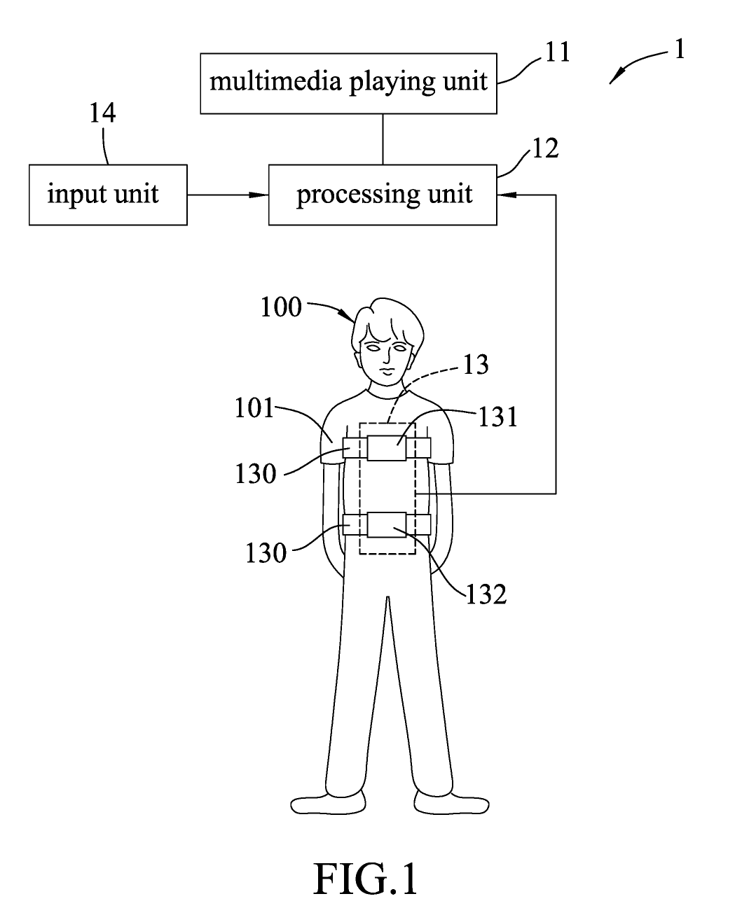

Referring to FIG. 1, a system 1 is used to implement the first embodiment of a method of estimating Internet activity dependence of a human subject 100 according to the disclosure. The system 1 includes a respiration sensing unit 13, a multimedia playing unit 11, an input unit 14, and a processing unit 12 coupled to the multimedia playing unit 11, the input unit 14 and the respiration sensing unit 13.

The respiration sensing unit 13 is, for example, attached to two belts 130 bonded around the torso 101 of the human subject 100 or to a smart garment (not shown) worn on the human subject 100. Herein, the respiration sensing unit 13 is wiredly connected to the processing unit 12. Alternatively, the respiration sensing unit 13 may communicate with the processing unit 12 using wireless communications. It is noted that the respiration sensing unit 13 includes a first respiration sensor 131 attached to the chest of the human subject 100 for sensing respiration-caused thorax movements of the human subject 100, and a second respiration sensor 132 attached to the abdomen of the human subject 100 for sensing respiration-caused abdomen movements of the human subject 100. The input unit 14 is manually operable by a user (not shown) to generate an input that is associated with an emotional stimulation material to be played on the multimedia playing unit 11. The multimedia playing unit 11 is controlled by the processing unit 12 to play the emotional stimulation material thereon. The emotional stimulation material may include either positive emotional content that tends to stimulate positive emotions in a viewer thereof or negative emotional content that tends to stimulate negative emotions in a viewer thereof.

In some embodiments, the multimedia playing unit 11, the processing unit 12 and the input unit 14, may be cooperatively implemented into, for example, a smart phone, a tablet computer, a personal computer or the like. The processing unit 12 may be installed with a software application (not shown) that includes instructions executable by the processing unit 12 to implement related steps of the method of the first embodiment of the disclosure.

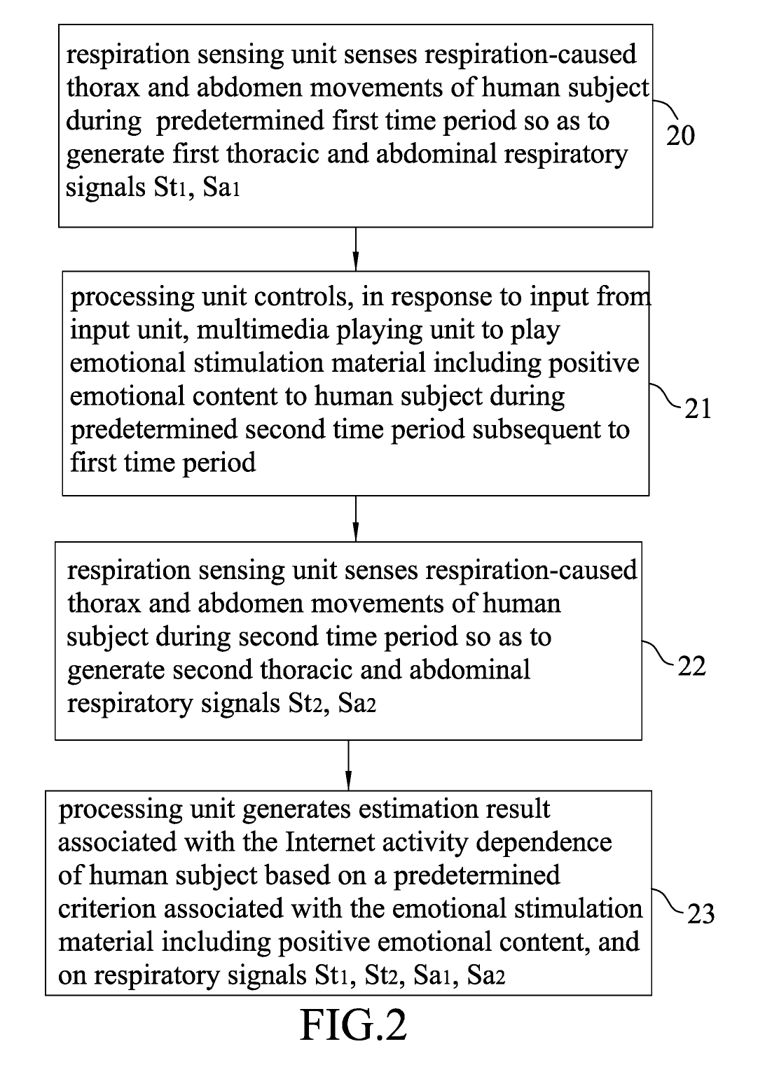

FIG. 2 is a flow chart illustrating the process of the first embodiment of the disclosure.

In step 20, the respiration sensing unit 13 senses respiration-caused thorax and abdomen movements of the human subject 100 during a predetermined first time period so as to generate one set of signals, including a first thoracic respiratory signal (St₁) and a first abdominal respiratory signal (Sa₁) and corresponding to the predetermined first time period.

In step 21, upon receipt of the input from the input unit 14, the processing unit 12 controls, in response to the input, the multimedia playing unit 11 to play the emotional stimulation material to the human subject 100 during a predetermined second time period subsequent to the predetermined first time period. The emotional stimulation material may be provided by the processing unit 12 or a storage medium (not shown) installed in the multimedia playing unit 11. In this embodiment, for example, the processing unit 12 provides, in response to the input from the input unit 14, the emotional stimulation material, which includes the positive emotional content, to the multimedia playing unit 11.

In step 22, as the emotional stimulation material displayed on the multimedia playing unit 11 is being displayed for view by the human subject 100, the respiration sensing unit 13 senses respiration-caused thorax and abdomen movements of the human subject 100 during the predetermined second time period so as to generate another set of signals, including a second thoracic respiratory signal (St₂) and a second abdominal respiratory signal (Sa₂) and corresponding to the predetermined second time period.

In step 23, the processing unit 12 generates an estimation result associated with the Internet activity dependence of the human subject 100 based on a predetermined criterion associated with the emotional stimulation material including the positive emotional content, on the first and second thoracic respiratory signals (St₁, St₂), and on the first and second abdominal respiratory signals (Sa₁, Sa₂).

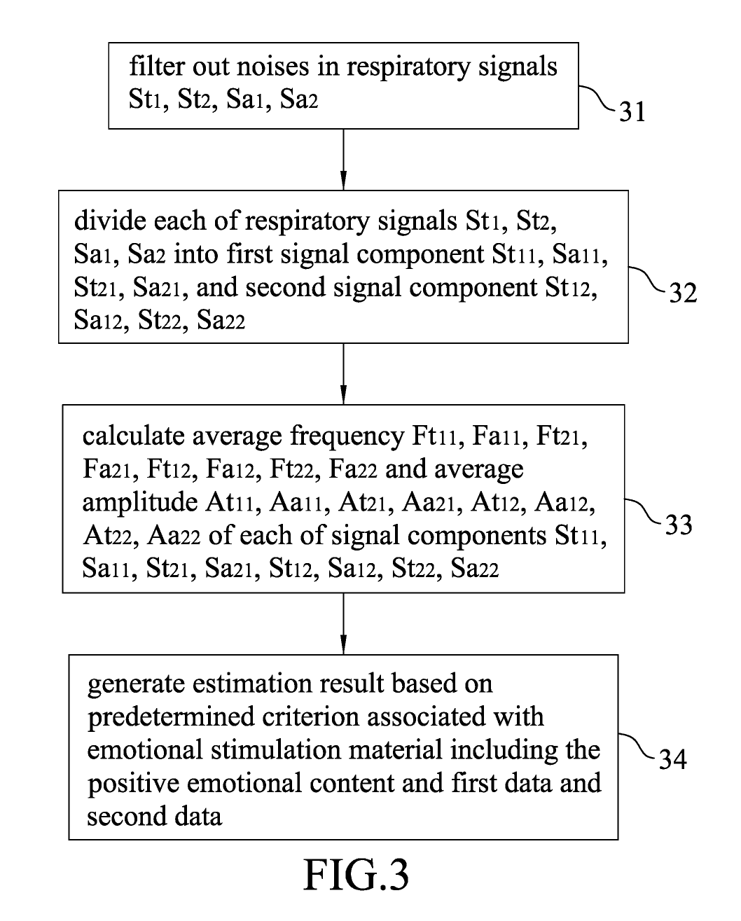

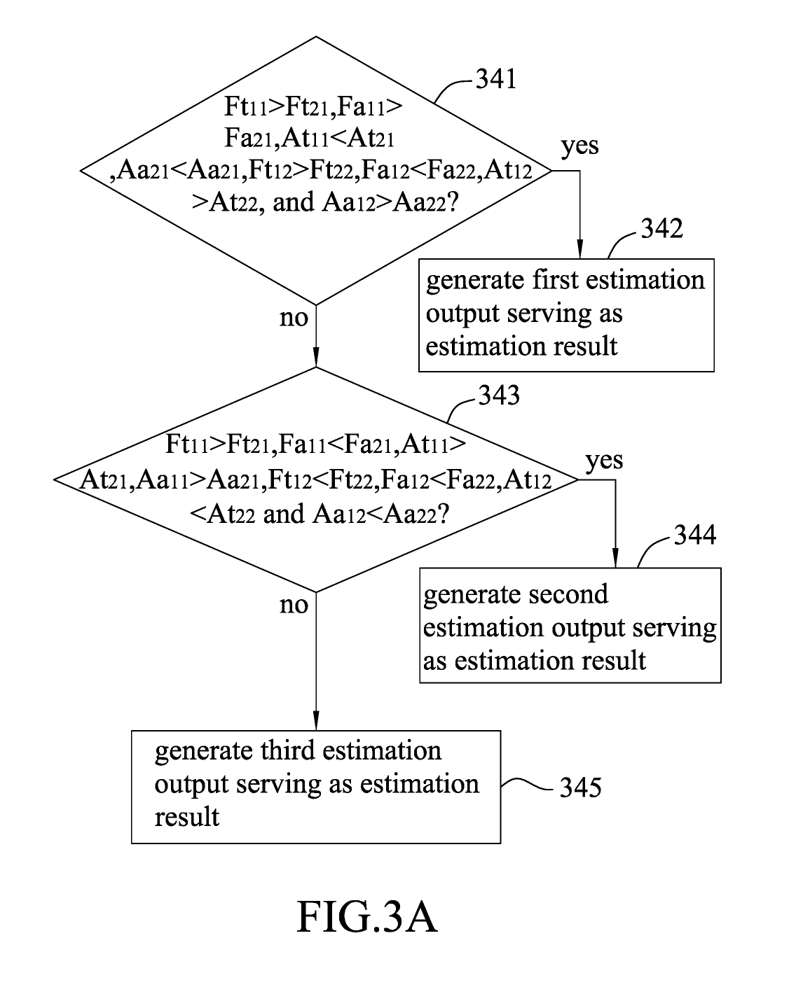

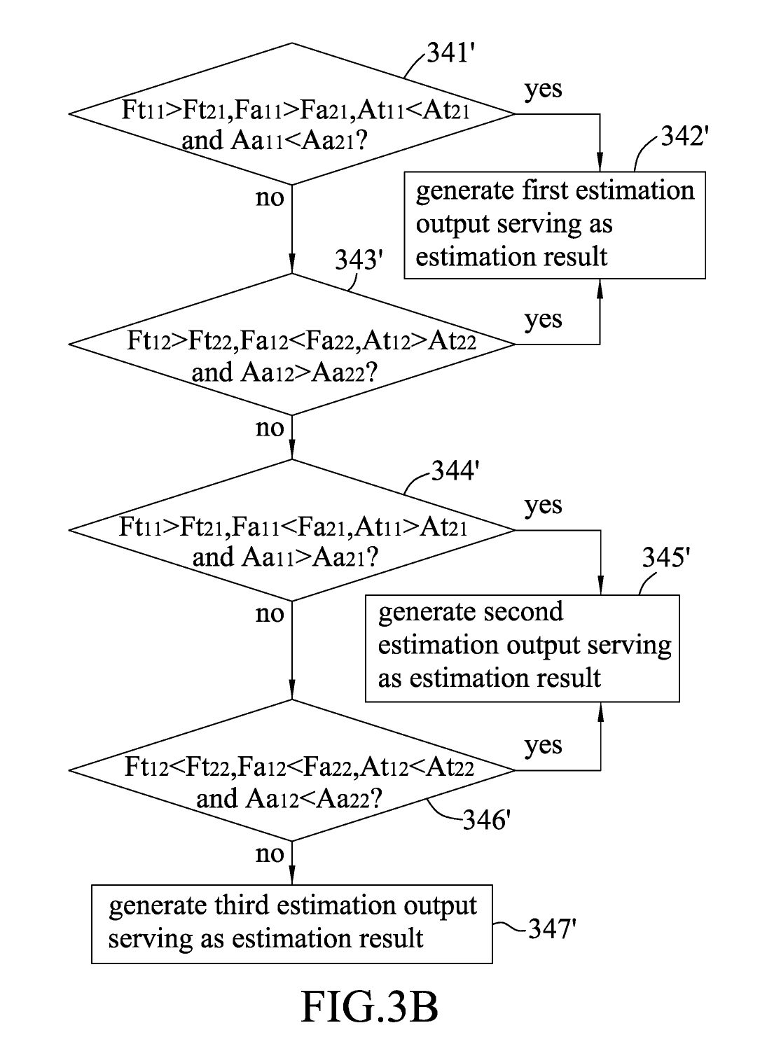

FIG. 3 is a flow chart illustrating how the processing unit 12 generates the estimation result based on the predetermined criterion associated with the emotional stimulation material including the positive emotional content, on the first and second thoracic respiratory signals (St₁, St₂), and on the first and second abdominal respiratory signals (Sa₁, Sa₂).

In step 31, the processing unit 12 filters out noises in the first and second thoracic respiratory signals respiratory signals (Sa₁, Sa₂). In this embodiment, the noises filtered out by the processing unit 12 have frequencies higher than, for example, 50 Hz or lower than, for example, 0.01 Hz.

In step 32, the processing unit 12 divides, using a decomposition method, the first thoracic respiratory signal (St₁) into a first signal component (St₁₁) and a second signal component (St₁₂) whose frequencies differ from each other, the first abdominal respiratory signal (Sa₁) into a first signal component (Sa₁₁) and a second signal component (Sa₁₂) whose frequencies differ from each other, the second thoracic respiratory signal (St₂) into a first signal component (St₂₁) and a second signal component (St₂₂) whose frequencies differ from each other, and the second abdominal respiratory signal (Sa₂) into a first signal component (Sa₂₁) and a second signal component (Sa₂₂) whose frequencies differ from each other. In this embodiment, the decomposition method is associated with, for example, a complementary ensemble empirical model proposed in an article by H. M. Ji, et al., entitled “Muscle cluster extraction of inspiratory movement by using complimentary ensemble empirical mode decomposition”. Since the feature of this disclosure does not reside in the decomposition method, details of the same are omitted herein for the sake of brevity.

In step 33, the processing unit 12 calculates an average frequency (Ft₁₁) and an average amplitude (At₁₁) of the first signal component (St₁₁), an average frequency (Ft₁₂) and an average amplitude (At₁₂) of the second signal component (St₁₂), an average frequency (Fa₁₁) and an average amplitude (Aa₁₁) of the first signal component (Sa₁₁), an average frequency (Fa₁₂) and an average amplitude (Aa₁₂) of the second signal component (Sa₁₂), an average frequency (Ft₂₁) and an average amplitude (At₂₁) of the first signal component (St₂₁), an average frequency (Ft₂₂) and an average amplitude At₂₂) of the second signal component (St₂₂), an average frequency (Fa₂₁) and an average amplitude (Aa₂₁) of the first signal component (Sa₂₁), and an average frequency (Fa₂₂) and an average amplitude (Aa₂₂) of the second signal component (Sa₂₂). In this embodiment, the average frequencies (Ft₁₁, Fa₁₁, Ft₂₁, Fa₂₁) and the average amplitudes (At₁₁, Aa₁₁, At₂₁, Aa₂₁) cooperatively constitute first data, and the average frequencies (Ft₁₂, Fa₁₂, Ft₂₂, Fa₂₂) and the average amplitudes (At₁₂, Aa₁₂, At₂₂, Aa₂₂) cooperatively constitute second data.

For example, the average amplitudes (At₁₁, At₁₂, At₂₁, At₂₂) and the average amplitudes (Aa₁₁, Aa₁₂, Aa₂₁, Aa₂₂) can be obtained respectively by the following equations (1) and (2):

$$\begin{matrix}{At}_{jk} = \frac{1}{s} {\sum }_{n = 1}^{s} {St}_{jk} ( n ) , 1 ≦ j , k ≦ 2 & ( 1 ) \\ {Aa}_{jk} = \frac{1}{s} {\sum }_{n = 1}^{s} {Sa}_{jk} ( n ) , 1 ≦ j , k ≦ 2 , & ( 2 )\end{matrix}$$

where s represents a sample size of each of the first and second signal components, Stⱼₖ(n) represents an amplitude of an n^{th} sample in the first/second signal component Stⱼₖ, Saⱼₖ(n) represents an amplitude of an n^{th} sample in the first/second signal component Saⱼₖ.

It is noted that average frequencies (Ft₁₁, Fa₁₁, Ft₂₁, Fa₂₁) of the first signal components (St₁₁, Sa₁₁, St₂₁, S₂₁) are all in a first frequency band, for example, higher than 1 Hz but not higher than 50 Hz. In addition, average frequencies (Ft₁₂, Fa₁₂, Ft₂₂, Fa₂₂) of the second signal components (St₁₂, Sa₁₂, St₂₂, Sa₂₂) are all in a second frequency band, for example, higher than 0.01 Hz but not higher than 1 Hz.

In step 34, the processing unit 12 generates, the estimation result based on the predetermined criterion associated with the emotional stimulation material including the positive emotional content, the first data and the second data.

FIG. 3A is an exemplary flow chart illustrating how the processing unit 12 generates the estimation result based on the predetermined criterion associated with the emotional stimulation material including the positive emotional content, the first data and the second data in the first embodiment.

In step 341, the processing unit 12 determines whether the average frequency (Ft₁₁) is greater than the average frequency (Ft₂₁), whether the average frequency (Fa₁₁) is greater than the average frequency (Fa₂₁), whether the average amplitude (At₁₁) is smaller than the average amplitude (At₂₁) and whether the average amplitude (Aa₁₁) is smaller than the average amplitude (Aa₂₁) so as to generate a first determination output. Furthermore, the processing unit 12 determines whether the average frequency (Ft₁₂) is greater than the average frequency (Ft₂₂), whether the average frequency (Fa₁₂) is smaller than the average frequency (Fa₂₂), whether the average amplitude (At₁₂) is greater than the average amplitude (At₂₂) and whether the average amplitude (Aa₁₂) is greater than the average amplitude (Aa₂₂) so as to generate a second determination output. When the first determination output generated by the processing unit 12 indicates that Ft₁₁>Ft₂₁, Fa₁₁>Fa₂₁, At₁₁<At₂₁ and Aa₁₁<Aa₂₁ while the second determination output generated by the processing unit 12 indicates that Ft₁₂>Ft₂₂, Fa₁₂<Fa₂₂, At₁₂>At₂₂ and Aa₁₂>Aa₂₂, the flow proceeds to step 342. Otherwise, the flow goes to step 343.

In step 342, the processing unit 12 generates a first estimation output which indicates that the human subject 100 is relatively highly dependent on Internet activity and which serves as the estimation result.

In step 343, the processing unit 12 determines whether the average frequency (Ft₁₁) is greater than the average frequency (Ft₂₁), whether the average frequency (Fa₁₁) is smaller than the average frequency (Fa₂₁), whether the average amplitude (At₁₁) is greater than the average amplitude (At₂₁) and whether the average amplitude (Aa₁₁) is greater than the average amplitude (Aa₂₁) so as to generate a third determination output. Furthermore, the processing unit 12 determines whether the average frequency (Ft₁₂) is smaller than the average frequency (Ft₂₂), whether the average frequency (Fa₁₂) is smaller than the average frequency (Fa₂₂), whether the average amplitude (At₁₂) is smaller than the average amplitude (At₂₂) and whether the average amplitude (Aa₁₂) is smaller than the average amplitude (Aa₂₂) so as to generate a fourth determination output. When the third determination output generated by the processing unit 12 indicates that Ft₁₁>Ft₂₁, Fa₁₁<Fa₂₁, Atₙ>At₂₁ and Aa₁₁>Aa₂₁ while the fourth determination output generated by the processing unit 12 indicates that Ft₁₂<Ft₂₂, Fa₁₂<Fa₂₂, At₁₂<At₂₂ and Aa₁₂<Aa₂₂, the flow proceeds to step 344. Otherwise, the flow goes to step 345.

In step 344, the processing unit 12 generates a second estimation output which indicates that the human subject 100 is relatively lowly dependent on Internet activity and which serves as the estimation result.

In this example, each of the first and third determination outputs can be regarded as a corresponding first comparison result associated with the average frequencies (Ft₁₁, Ft₂₁, Fa₁₁, Fa₂₂) and the average amplitudes (At₁₁, At₂₁, Aa₁₁, Aa₂₁) Each of the second and fourth determination outputs can be regarded as a corresponding second comparison result associated with the average frequencies (Ft₁₂, Ft₂₂, Fa₁₂, Fa₂₂) and the average amplitudes (At₁₂, At₂₂, Aa₁₂, Aa₂₂).

In step 345, the processing unit 12 generates a third estimation output indicating inconclusive result as to Internet activity dependence of the human subject 100 and serving as the estimation result.

Therefore, Table 1 built in accordance with this example of FIG. 3A can serve as a lookup table that is adopted by the processing unit 12 to estimate the Internet activity dependence of the human subject 100 in the first embodiment.

TABLE 1

| | | positive emotional | | | |
|---|---|---|---|---|---|
| | | content | | | |
| | | average | | average | |
| | | frequency | | amplitude | |
| | | thorax | abdomen | thorax | abdomen |
| first | first | Ft₁₁ > | Fa₁₁ > | At₁₁ < | Aa₁₁ < |
| data | estimation | Ft₂₁ | Fa₂₁ | At₂₁ | Aa₂₁ |
| second | output | Ft₁₂ > | Fa₁₂ < | At₁₂ > | Aa₁₂ > |
| data | (relatively | Ft₂₂ | Fa₂₂ | At₂₂ | Aa₂₂ |
| | highly | | | | |
| | dependent) | | | | |
| first | second | Ft₁₁ > | Fa₁₁ < | At₁₁ > | Aa₁₁ > |
| data | estimation | Ft₂₁ | Fa₂₁ | At₂₁ | Aa₂₁ |
| second | output | Ft₁₂ < | Fa₁₂ < | At₁₂ < | Aa₁₂ < |
| data | (relatively | Ft₂₂ | Fa₂₂ | At₂₂ | Aa₂₂ |
| | lowly | | | | |
| | dependent) | | | | |

FIG. 3B is another exemplary flow chart illustrating how the processing unit 12 generates the estimation result based on the predetermined criterion associated with the emotional stimulation material including the positive emotional content, the first data and the second data in the first embodiment.

In step 341′, the processing unit 12 determines whether the average frequency (Ft₁₁) is greater than the average frequency (Ft₂₁), whether the average frequency (Fa₁₁) is greater than the average frequency (Fa₂₁), whether the average amplitude (At₁₁) is smaller than the average amplitude (At₂₁) and whether the average amplitude (Aa₁₁) is smaller than the average amplitude (Aa₂₁) so as to generate a first determination output. When the first determination output generated by the processing unit 12 indicates that Ft₁₁>Ft₂₁, Fa₁₁>Fa₂₁, At₁₁<At₂₁ and Aa₁₁<Aa₂₁, the flow proceeds to step 342′. Otherwise, the flow goes to step 343′.

In step 342′, similar to step 342 of FIG. 3A, the processing unit 12 generates the first estimation output serving as the estimation result.

In step 343′, the processing unit 12 determines whether the average frequency (Ft₁₂) is greater than the average frequency (Ft₂₂), whether the average frequency (Fa₁₂) is smaller than the average frequency (Fa₂₂), whether the average amplitude (At₁₂) is greater than the average amplitude (At₂₂) and whether the average amplitude (Aa₁₂) is greater than the average amplitude (Aa₂₂) so as to generate a second determination output. When the second determination output generated by the processing unit 12 indicates that Ft₁₂>Ft₂₂, Fa₁₂<Fa₂₂, At₁₂>At₂₂ and Aa₁₂>Aa₂₂, the flow goes back to step 342′. Otherwise, the flow proceeds to step 344′.

In step 344′, the processing unit 12 determines whether the average frequency (Ft₁₁) is greater than the average frequency (Ft₂₁), whether the average frequency (Fa₁₁) is smaller than the average frequency (Fa₂₁), whether the average amplitude (At₁₁) is greater than the average amplitude (At₂₁) and whether the average amplitude (Aa₁₁) is greater than the average amplitude (Aa₂₁) so as to generate a third determination output. When the third determination output generated by the processing unit 12 indicates that Ft₁₁>Ft₂₁, Fa₁₁<Fa₂₁, At₁₁>At₂₁ and Aa₁₁>Aa₂₁, the flow proceeds to step 345′. Otherwise, the flow goes to step 346′.

In step 345′, similar to step 344 of FIG. 3A, the processing unit 12 generates the second estimation output serving as the estimation result.

In step 346′, the processing unit 12 determines whether the average frequency (Ft₁₂) is smaller than the average frequency (Ft₂₂), whether the average frequency (Fa₁₂) is smaller than the average frequency (Fa₂₂), whether the average amplitude (At₁₂) is smaller than the average amplitude (At₂₂) and whether the average amplitude (Aa₁₂) is smaller than the average amplitude (Aa₂₂) so as to generate a fourth determination output. When the fourth determination output generated by the processing unit 12 indicates that Ft₁₂<Ft₂₂, Fa₁₂<Fa₂₂, At₁₂<At₂₂ and Aa₁₂<Aa₂₂, the flow goes back to step 345′. Otherwise, the flow proceeds to step 347′.

In this example, similar to the example shown in FIG. 3A, each of the first and third determination outputs can be regarded as a corresponding first comparison result associated with the average frequencies (Ft₁₁, Ft₂₁, Fa₁₁, Fa₂₁) and the average amplitudes (At₁₁, At₂₁, Aa₁₁, Aa₂₁) Each of the second and fourth determination outputs can be regarded as a corresponding second comparison result associated with the average frequencies (Ft₁₂, Ft₂₂, Fa₁₂, Fa₂₂) and the average amplitudes (At₁₂, At₂₂, Aa₁₂, Aa₂₂).

In step 347′, similar to step 345 of FIG. 3A, the processing unit 12 generates the third estimation output serving as the estimation result.

Therefore, Table 2 built in accordance with this example of FIG. 3B can serve as another lookup table that is adopted by the processing unit 12 to estimate the Internet activity dependence of the human subject 100 in the first embodiment.

TABLE 2

| | | positive emotional | | | |
|---|---|---|---|---|---|
| | | content | | | |
| | | average | | average | |
| | | frequency | | amplitude | |
| | | thorax | abdomen | thorax | abdomen |
| first | first | Ft₁₁ > | Fa₁₁ > | At₁₁ < | Aa₁₁ < |
| data | estimation | Ft₂₁ | Fa₂₁ | At₂₁ | Aa₂₁ |
| | output | | | | |
| | (relatively | | | | |
| | highly | | | | |
| | dependent) | | | | |
| | second | Ft₁₁ > | Fa₁₁ < | At₁₁ > | Aa₁₁ > |
| | estimation | Ft₂₁ | Fa₂₁ | At₂₁ | Aa₂₁ |
| | output | | | | |
| | (relatively | | | | |
| | lowly | | | | |
| | dependent) | | | | |
| second | first | Ft₁₂ > | Fa₁₂ < | At₁₂ > | Aa₁₂ > |
| data | estimation | Ft₂₂ | Fa₂₂ | At₂₂ | Aa₂₂ |
| | output | | | | |
| | (relatively | | | | |
| | highly | | | | |
| | dependent) | | | | |
| | second | Ft₁₂ < | Fa₁₂ < | At₁₂ < | Aa₁₂ < |
| | estimation | Ft₂₂ | Fa₂₂ | At₂₂ | Aa₂₂ |
| | output | | | | |
| | (relatively | | | | |
| | lowly | | | | |
| | dependent) | | | | |

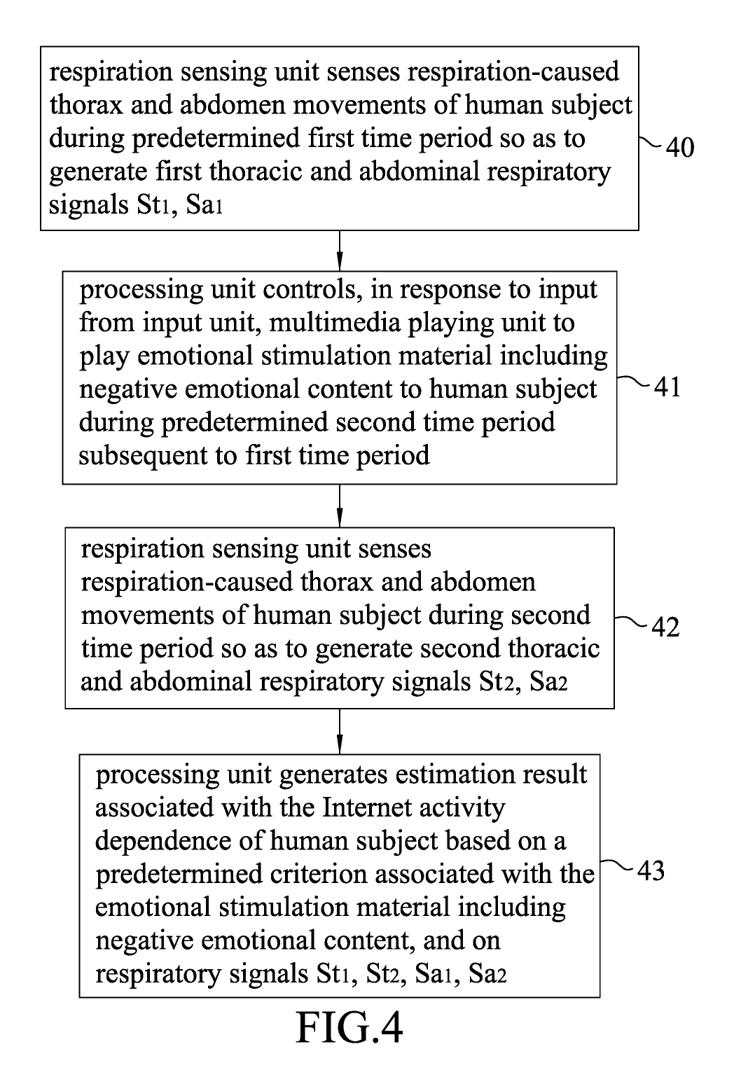

FIG. 4 is a flow chart illustrating how the system of FIG. 1 implements the second embodiment of a method of estimating Internet activity dependence of a human subject 100 according to the disclosure. The process of the second embodiment is a modification of that of the first embodiment of FIG. 2. Steps 40, 42 and 43 are similar respectively to steps 20, 22 and 23 of the first embodiment (FIG. 2).

However, in step 41, unlike step 21 of the first embodiment (FIG. 2), the processing unit 12 controls, in response to the input from the input unit 14, the multimedia playing unit 11 to play the emotional stimulation material that includes the negative emotional content to the human subject 100 during the predetermine second time period.

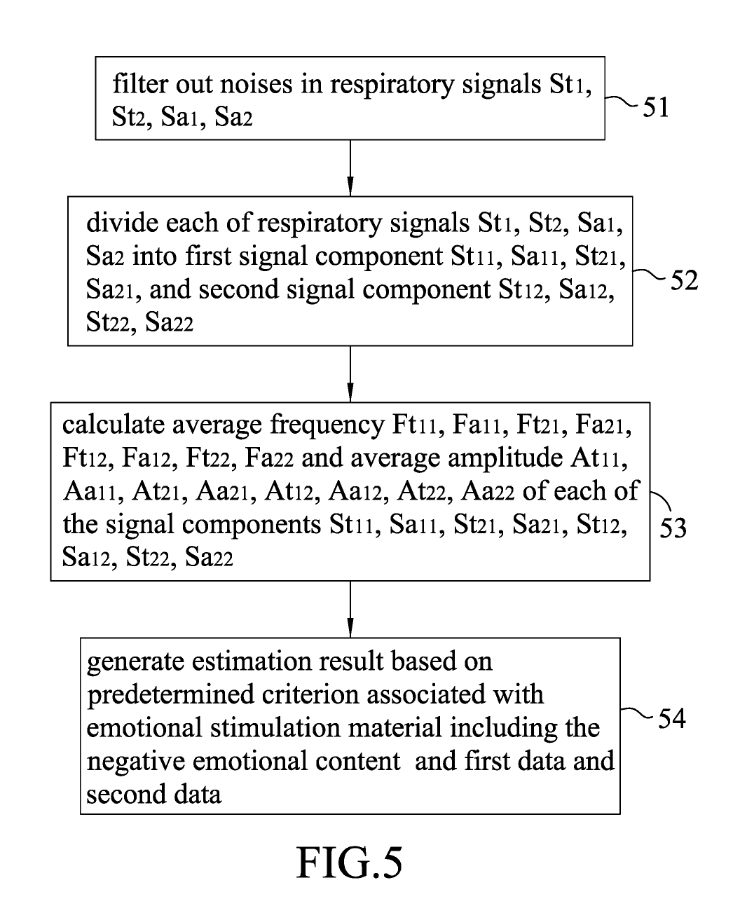

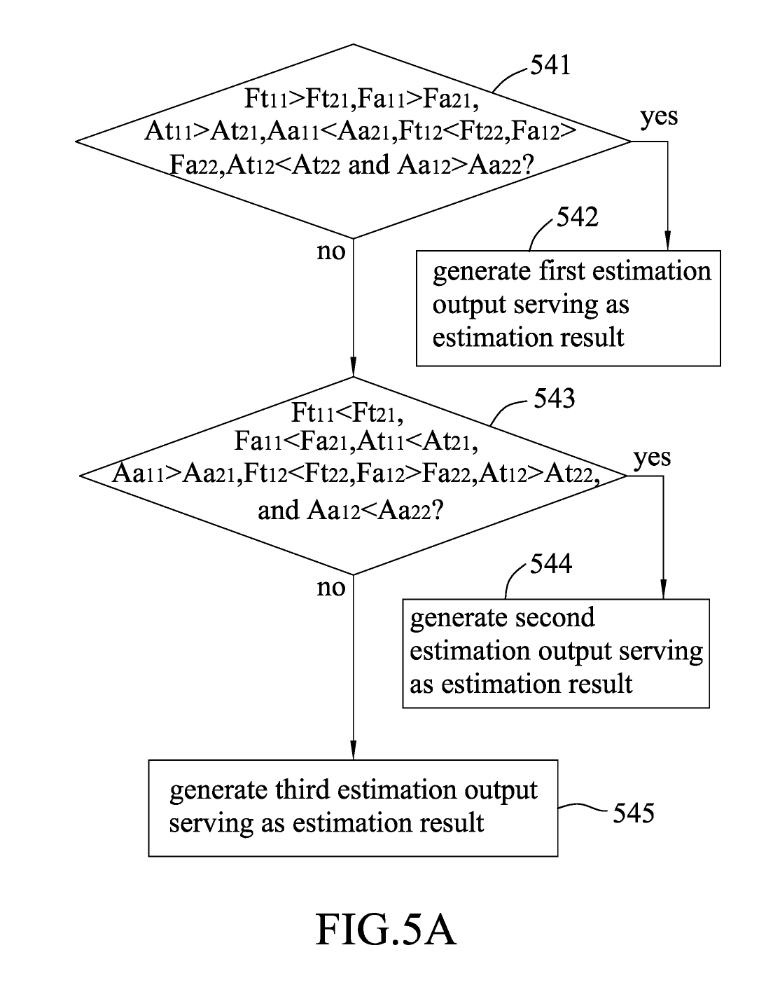

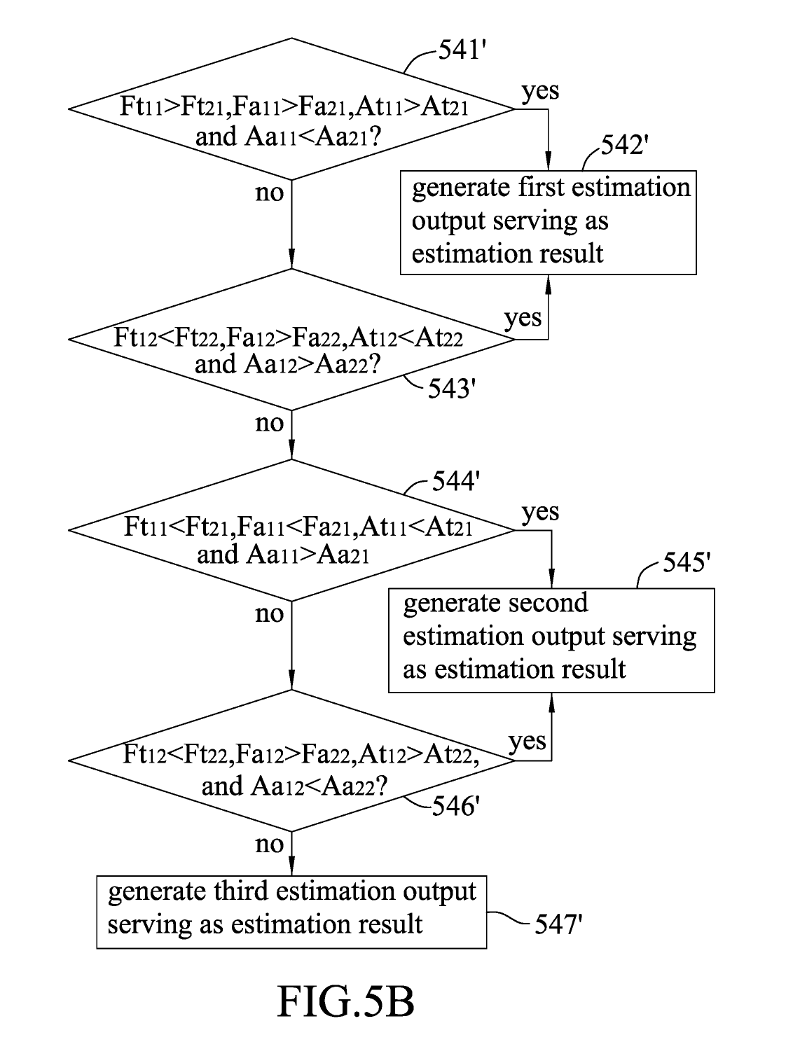

FIG. 5 is a flow chart illustrating how the processing unit 12 generates an estimation result based on a predetermined criterion associated with the emotional stimulation material including the negative emotional content, on the first and second thoracic respiratory signals (St₁, St₂), and on the first and second abdominal respiratory signals (Sa₁, Sa₂). In this embodiment, steps 51, 52 and 53 are similar respectively to steps 31, 32 and 33 of the first embodiment (FIG. 3). However, in step 54, unlike step 34 of the first embodiment (FIG. 3), the processing unit 12 generates the estimation result based on the predetermined criterion associated with the emotional stimulation material including the negative emotional content, the first data and the second data.

FIG. 5A is an exemplary flow chart illustrating how the processing unit 12 generates the estimation result based on the predetermined criterion associated with the emotional stimulation material including the negative emotional content, the first data and the second data in the second embodiment.

In step 541, the processing unit 12 determines whether the average frequency (Ft₁₁) is greater than the average frequency (Ft₂₁), whether the average frequency (Fa₁₁) is greater than the average frequency (Fa₂₁), whether the average amplitude (At₁₁) is greater than the average amplitude (At₂₁) and whether the average amplitude (Aa₁₁) is smaller than the average amplitude (Aa₂₁) so as to generate a first determination output. Furthermore, the processing unit 12 determines whether the average frequency (Ft₁₂) is smaller than the average frequency (Ft₂₂), whether the average frequency (Fa₁₂) is greater than the average frequency (Fa₂₂), whether the average amplitude (At₁₂) is smaller than the average amplitude (At₂₂) and whether the average amplitude (Aa₁₂) is greater than the average amplitude (Aa₂₂) so as to generate a second determination output. When the first determination output generated by the processing unit 12 indicates that Ft₁₁>Ft₂₁, Fa₁₁>Fa₂₁, A₁₁>At₂₁ and Aa₁₁<Aa₂₁ while the second determination output generated by the processing unit 12 indicates that Ft₁₂<Ft₂₂, Fa₁₂>Fa₂₂, At₁₂<At₂₂ and Aa₁₂>Aa₂₂, the flow proceeds to step 542. Otherwise, the flow goes to step 543.

In step 542, similar to step 342 of the first embodiment (FIG. 3A), the processing unit 12 generates the first estimation output serving as the estimation result.

In step 543, the processing unit 12 determines whether the average frequency (Ft₁₁) is smaller than the average frequency (Ft₂₁), whether the average frequency (Fa₁₁) is smaller than the average frequency (Fa₂₁), whether the average amplitude (At₁₁) is smaller than the average amplitude (At₂₁) and whether the average amplitude (Aa₁₁) is greater than the average amplitude (Aa₂₁) so as to generate a third determination output. Furthermore, the processing unit 12 determines whether the average frequency (Ft₁₂) is smaller than the average frequency (Ft₂₂), whether the average frequency (Fa₁₂) is greater than the average frequency (Fa₂₂), whether the average amplitude (At₁₂) is greater than the average amplitude (At₂₂) and whether the average amplitude (Aa₁₂) is smaller than the average amplitude (Aa₂₂) so as to generate a fourth determination output. When the third determination output generated by the processing unit 12 indicates that Ft₁₁<Ft₂₁, Fa₁₁<Fa₂₁, A₁₁<At₂₁ and Aa₁₁>Aa₂₁ while the fourth determination output generated by the processing unit 12 indicates that Ft₁₂<Ft₂₂, Fa₁₂>Fa₂₂, At₁₂>At₂₂ and Aa₁₂<Aa₂₂, the flow proceeds to step 544. Otherwise, the flow goes to step 545.

In step 544, similar to step 344 of the first embodiment (FIG. 3A), the processing unit 12 generates the second estimation output serving as the estimation result.

In this example, similar to the example of FIG. 3A, each of the first and third determination outputs can be regarded as a corresponding first comparison result associated with the average frequencies (Ft₁₁, Ft₂₁, Fa₁₁, Fa₂₁) and the average amplitudes (At₁₁, At₂₁, Aa₁₁, Aa₂₁). Each of the second and fourth determination outputs can be regarded as a corresponding second comparison result associated with the average frequencies (Ft₁₂, Ft₂₂, Fa₁₂, Fa₂₂) and the average amplitudes (At₁₂, At₂₂, Aa₁₂, Aa₂₂).

In step 545, similar to step 343 of the first embodiment (FIG. 3A), the processing unit 12 generates the third estimation output serving as the estimation result.

Therefore, Table 3 built in accordance with this example of FIG. 5A can serve as a lookup table that is adopted by the processing unit 12 to estimate the Internet activity dependence of the human subject 100 in the second embodiment.

TABLE 3

| | | negative emotional | | | |
|---|---|---|---|---|---|
| | | content | | | |
| | | average | | average | |
| | | frequency | | amplitude | |
| | | thorax | abdomen | thorax | abdomen |
| first | first | Ft₁₁ > | Fa₁₁ > | At₁₁ > | Aa₁₁ < |
| data | estimation | Ft₂₁ | Fa₂₁ | At₂₁ | Aa₂₁ |
| second | output | Ft₁₂ < | Fa₁₂ > | At₁₂ < | Aa₁₂ > |
| data | (relatively | Ft₂₂ | Fa₂₂ | At₂₂ | Aa₂₂ |
| | highly | | | | |
| | dependent) | | | | |
| first | second | Ft₁₁ < | Fa₁₁ < | At₁₁ < | Aa₁₁ > |
| data | estimation | Ft₂₁ | Fa₂₁ | At₂₁ | Aa₂₁ |
| second | output | Ft₁₂ < | Fa₁₂ > | At₁₂ > | Aa₁₂ < |
| data | (relatively | Ft₂₂ | Fa₂₂ | At₂₂ | Aa₂₂ |
| | lowly | | | | |
| | dependent) | | | | |

FIG. 5B is another exemplary flow chart illustrating how the processing unit 12 generates the estimation result based on the predetermined criterion associated with the emotional stimulation material including the negative emotional content, the first data and the second data in the second embodiment.

In step 541′, the processing unit 12 determines whether the average frequency (Ft₁₁) is greater than the average frequency (Ft₂), whether the average frequency (Fa₁₁) is greater than the average frequency (Fa₂₁), whether the average amplitude (At₁₁) is greater than the average amplitude (At₂₁) and whether the average amplitude (Aa₁₁) is smaller than the average amplitude (Aa₂₁) so as to generate a first determination output. When the first determination output generated by the processing unit 12 indicates that Ft₁₁>Ft₂₁, Fa₁₁>Fa₂₁, At₁₁>At₂₁ and Aa₁₁<Aa₂₁, the flow proceeds to step 542′. Otherwise, the flow goes to step 543′.

In step 542′, similar to step 342′ of the first embodiment (FIG. 3B), the processing unit 12 generates the first estimation output serving as the estimation result.

In step 543′, the processing unit 12 determines whether the average frequency (Ft₁₂) is smaller than the average frequency (Ft₂₂), whether the average frequency (Fa₁₂) is greater than the average frequency (Fa₂₂), whether the average amplitude (At₁₂) is smaller than the average amplitude (At₂₂) and whether the average amplitude (Aa₁₂) is greater than the average amplitude (Aa₂₂) so as to generate a second determination output. When the second determination output generated by the processing unit 12 indicates that Ft₁₂<Ft₂₂, Fa₁₂>Fa₂₂, At₁₂<At₂₂ and Aa₁₂>Aa₂₂, the flow goes back to step 542′. Otherwise, the flow proceeds to step 544′.

In step 544′, the processing unit 12 determines whether the average frequency (Ft₁₁) is smaller than the average frequency (Ft₂₁), whether the average frequency (Fa₁₁) is smaller than the average frequency (Fa₂₁), whether the average amplitude (At₁₁) is smaller than the average amplitude (At₂₁) and whether the average amplitude (Aa₁₁) is greater than the average amplitude (Aa₂₁) so as to generate a third determination output. When the third determination output generated by the processing unit 12 indicates that Ft₁₁<Ft₂₁, Fa₁₁<Fa₂₁, At₁₁<At₂₁ and Aa₁₁>Aa₂₁, the flow proceeds to step 545′. Otherwise, the flow goes to step 546′.

In step 545′, similar to step 345′ of the first embodiment (FIG. 3B), the processing unit 12 generates the second estimation output serving as the estimation result.

In step 546′, the processing unit 12 determines whether the average frequency (Ft₁₂) is smaller than the average frequency (Ft₂₂) whether the average frequency (Fa₁₂) is greater than the average frequency (Fa₂₂), whether the average amplitude (At₁₂) is greater than the average amplitude (At₂₂) and whether the average amplitude (Aa₁₂) is smaller than the average amplitude (Aa₂₂) so as to generate a fourth determination output. When the fourth determination output generated by the processing unit 12 indicates that Ft₁₂<Ft₂₂, Fa₁₂>Fa₂₂, At₁₂>At₂₂ and Aa₁₂<Aa₂₂, the flow goes back to step 545′. Otherwise, the flow proceeds to step 547′.

In this example, similar to the example shown in FIG. 5A, each of the first and third determination outputs can be regarded as a corresponding first comparison result associated with the average frequencies (Ft₁₁, Ft₂₁, Fa₁₁, Fa₂₁) and the average amplitudes (At₁₁, At₂₁, Aa₁₁, Aa₂₁) Each of the second and fourth determination outputs can be regarded as a corresponding second comparison result associated with the average frequencies (Ft₁₂, Ft₂₂, Fa₁₂, Fa₂₂) and the average amplitudes (At₁₂, At₂₂, Aa₁₂, Aa₂₂).

In step 547′, similar to step 347′ of the first embodiment (FIG. 3B), the processing unit 12 generates the third estimation output serving as the estimation result.

Therefore, Table 4 built in accordance with this example of FIG. 5B can serve as another lookup table that is adopted by the processing unit 12 to estimate the Internet activity dependence of the human subject 100 in the second embodiment.

TABLE 4

| | | negative emotional | | | |
|---|---|---|---|---|---|
| | | content | | | |
| | | average | | average | |
| | | frequency | | amplitude | |
| | | thorax | abdomen | thorax | abdomen |
| first | first | Ft₁₁ > | Fa₁₁ > | At₁₁ > | Aa₁₁ < |
| data | estimation | Ft₂₁ | Fa₂₁ | At₂₁ | Aa₂₁ |
| | output | | | | |
| | (relatively | | | | |
| | highly | | | | |
| | dependent) | | | | |
| | second | Ft₁₁ < | Fa₁₁ < | At₁₁ < | Aa₁₁ > |
| | estimation | Ft₂₁ | Fa₂₁ | At₂₁ | Aa₂₁ |
| | output | | | | |
| | (relatively | | | | |
| | lowly | | | | |
| | dependent) | | | | |
| second | first | Ft₁₂ < | Fa₁₂ > | At₁₂ < | Aa₁₂ > |
| data | estimation | Ft₂₂ | Fa₂₂ | At₂₂ | Aa₂₂ |
| | output | | | | |
| | (relatively | | | | |
| | highly | | | | |
| | dependent) | | | | |
| | second | Ft₁₂ < | Fa₁₂ > | At₁₂ > | Aa₁₂ < |
| | estimation | Ft₂₂ | Fa₂₂ | At₂₂ | Aa₂₂ |
| | output | | | | |
| | (relatively | | | | |
| | lowly | | | | |
| | dependent) | | | | |

It is noted that partially combining the first and second embodiments may result in the third embodiment of a method of estimating Internet activity dependence of a human subject 100 according to the disclosure.

The following describes the process of the third embodiment implemented by the system of FIG. 1.

For example, firstly, steps 20, 21 and 22 of the first embodiment (FIG. 2) are sequentially performed, thereby generating a first set of signals including a first thoracic respiratory signal (St₁) and a first abdominal respiratory signal (Sa₁), and a second set of signals including a second thoracic respiratory signal (St₂) and a second abdominal respiratory signal (Sa₂) and associated with the emotional stimulation material including the positive emotional content. After a while, for example, 3 minutes or longer, steps 41 and 42 of the second embodiment (FIG. 4) are sequentially performed, thereby generating a third set of signals including a second thoracic respiratory signal (St₂′) and a second abdominal respiratory signal (Sa₂′) and associated with another emotional stimulation material including the negative emotional content.

Thus, the processing unit 12 generates an estimation result associated with the Internet activity dependence of the human subject 100 based on the predetermined criterion associated with the emotional stimulation material including the positive emotional content, the predetermined criterion associated with the emotional stimulation material including the negative emotional content, on the thoracic respiratory signals (St₁, St₂, St₂′), and on the abdominal respiratory signals (Sa₁, Sa₂, Sa₂′).

In this embodiment, similar to steps 31, 32 and 33 of the first embodiment (FIG. 3) or steps 51, 52 and 53 of the second embodiment (FIG. 5), the processing unit 12 filters out noises in the thoracic respiratory signals (St₁, St₂, St₂′) and the abdominal respiratory signals (Sa₁, Sa₂, Sa₂′), divides each of the thoracic respiratory signals (St₁, St₂, St₂′) and the abdominal respiratory signals (Sa₁, Sa₂, Sa₂′) into a first signal component (St₁₁, St₂₁, St₂₁′, Sa₁₁, Sa₂₁, Sa₂₁′) and a second signal component (St₁₂, St₂₂, St₂₂′, Sa₁₂, Sa₂₂, Sa₂₂′) using the aforesaid decomposition method, and calculates an average frequency (Ft₁₁, Ft₂₁, Ft₂₁′, Fa₁₁, Fa₂₁, Fa₂₁′, Ft₁₂, Ft₂₂, Ft₂₂′, Fa₁₂, Fa₂₂, Fa₂₂′) and an average amplitude (At₁₁, At₂₁, At₂₁′, Aa₁₁, Aa₂₁, Aa₂₁′, At₁₂, At₂₂, At₂₂′, Aa₁₂, Aa₂₂, Aa₂₂′) of each of the first and second signal components (St₁₁, St₂₁, St₂₁′, Sa₁₁, Sa₂₁, Sa₂₁′, St₁₂, St₂₂, St₂₂′, Sa₁₂, Sa₂₂, Sa₂₂′). It is noted that the average frequencies (Ft₁₁, Fa₁₁, Ft₂₁, Fa₂₁, Ft₂₁′, Fa₂₁′) and the average amplitudes (At₁₁, Aa₁₁, At₂₁, Aa₂₁, At₂₁, Aa₂₁) cooperatively constitute first data, and the average frequencies (Ft₁₂, Fa₁₂, Ft₂₂, Fa₂₂, Ft₂₂′, Fa₂₂′) and the average amplitudes (At₁₂, Aa₁₂, At₂₂, Aa₂₂, At₂₂′, Aa₂₂′) cooperatively constitute second data.

Therefore, Table 5, which is generated based on Tables 1 and 3, can serve as an exemplary lookup table that is adopted by the processing unit 12 to estimate the Internet activity dependence of the human subject 100 in the third embodiment.

TABLE 5

| | | positive | | negative | |
|---|---|---|---|---|---|
| | | emotional | | emotional | |
| | | content | | content | |
| | | thorax | abdomen | thorax | abdomen |
| first | first | average frequency | | | |
| data | estimation | Ft₁₁ > | Fa₁₁ > | Ft₁₁ > | Fa₁₁ > |
| | output | Ft₂₁ | Fa₂₁ | Ft₂₁′ | Fa₂₁′ |
| | (relatively | average amplitude | | | |
| | highly | At₁₁ < | Aa₁₁ < | At₁₁ > | Aa₁₁ < |
| | dependent) | At₂₁ | Aa₂₁ | At₂₁′ | Aa₂₁′ |
| second | | average frequency | | | |
| data | | Ft₁₂ > | Fa₁₂ < | Ft₁₂ < | Fa₁₂ > |
| | | Ft₂₂ | Fa₂₂ | Ft₂₂′ | Fa₂₂′ |
| | | average amplitude | | | |
| | | At₁₂ > | Aa₁₂ > | At₁₂ < | Aa₁₂ > |
| | | At₂₂ | Aa₂₂ | At₂₂′ | Aa₂₂′ |
| first | second | average frequency | | | |
| data | estimation | Ft₁₁ > | Fa₁₁ < | Ft₁₁ < | Fa₁₁ < |
| | output | Ft₂₁ | Fa₂₁ | Ft₂₁′ | Fa₂₁′ |
| | (relatively | average amplitude | | | |
| | lowly | At₁₁ > | Aa₁₁ > | At₁₁ < | Aa₁₁ > |
| | dependent) | At₂₁ | Aa₂₁ | At₂₁′ | Aa₂₁′ |
| second | | average frequency | | | |
| data | | Ft₁₂ < | Fa₁₂ < | Ft₁₂ < | Fa₁₂ > |
| | | Ft₂₂ | Fa₂₂ | Ft₂₂′ | Fa₂₂′ |

| average amplitude | | | |
|---|---|---|---|
| At₁₂ < | Aa₁₂ < | At₁₂ > | Aa₁₂ < |
| At₂₂ | Aa₂₂ | At₂₂′ | Aa₂₂′ |

In addition, Table 6, which is generated based on Tables 2 and 4, can serve as another exemplary lookup table that is adopted by the processing unit 12 to estimate the Internet activity dependence of the human subject 100 in the third embodiment.

TABLE 6

| | | positive | | negative | |
|---|---|---|---|---|---|
| | | emotional | | emotional | |
| | | content | | content | |
| | | thorax | abdomen | thorax | abdomen |
| first | first | average frequency | | | |
| data | estimation | Ft₁₁ > | Fa₁₁ > | Ft₁₁ > | Fa₁₁ > |
| | output | Ft₂₁ | Fa₂₁ | Ft₂₁′ | Fa₂₁′ |
| | (relatively | average amplitude | | | |
| | highly | At₁₁ < | Aa₁₁ < | At₁₁ > | Aa₁₁ < |
| | dependent) | At₂₁ | Aa₂₁ | At₂₁′ | Aa₂₁′ |
| | second | average frequency | | | |
| | estimation | Ft₁₁ > | Fa₁₁ < | Ft₁₁ < | Fa₁₁ < |
| | output | Ft₂₁ | Fa₂₁ | Ft₂₁′ | Fa₂₁′ |
| | (relatively | average amplitude | | | |
| | lowly | At₁₁ > | Aa₁₁ > | At₁₁ < | Aa₁₁ > |
| | dependent) | At₂₁ | Aa₂₁ | At₂₁′ | Aa₂₁′ |
| second | first | average frequency | | | |
| data | estimation | Ft₁₂ > | Fa₁₂ < | Ft₁₂ < | Fa₁₂ > |
| | output | Ft₂₂ | Fa₂₂ | Ft₂₂′ | Fa₂₂ |
| | (relatively | average amplitude | | | |
| | highly | At₁₂ > | Aa₁₂ > | At₁₂ < | Aa₁₂ > |
| | dependent) | At₂₂ | Aa₂₂ | At₂₂′ | Aa₂₂′ |
| | second | average frequency | | | |
| | estimation | Ft₁₂ < | Fa₁₂ < | Ft₁₂ < | Fa₁₂ > |
| | output | Ft₂₂ | Fa₂₂ | Ft₂₂′ | Fa₂₂′ |
| | (relatively | average amplitude | | | |
| | lowly | At₁₂ < | Aa₁₂ < | At₁₂ > | Aa₁₂ < |
| | dependent) | At₂₂ | Aa₂₂ | At₂₂′ | Aa₂₂′ |

In Tables 5 and 6, each of the first and second estimation outputs generated by the processing unit 12 can serve as the estimation result. When other comparison results associated with the first and second data are not listed in Table 5 or Table 6, similar to steps 345 and 347′ of the first embodiment (FIGS. 3A and 3B) and steps 545 and 547′ of the second embodiment (FIGS. 5A and 5B), the processing unit 12 generates the third estimation output indicating inconclusive result as to as to Internet activity dependence of the human subject 100 and serving as the estimation result.

In this embodiment, steps 21 and 22 of the first embodiment (FIG. 2) are performed prior to steps 41 and 42 of the second embodiment (FIG. 4). However, in some embodiments, steps 41 and 42 of the second embodiment (FIG. 4) may be performed prior to steps 21 and 22 of the first embodiment (FIG. 2).

In view of the above, the processing unit 12 can easily generate the estimation result associated with the Internet activity dependence of the human subject 100 based on the predetermined criterion associated with one emotional stimulation material including positive or negative emotional content or the predetermined criterion associated with each of two emotional stimulation materials respectively including positive and negative contents, and on the corresponding respiratory signal (St₁, St₂, St₂′, Sa₁, Sa₂, Sa₂′) sensed from the subject human 100 during the predetermined first and second time periods without filling out the time-consuming questionnaires as required in the prior art. Therefore, a current estimation result of the subject human 100 can be obtained using the method of the disclosure in a relatively short amount of time, for example, several minutes, and may be used as reference information by a doctor to diagnose whether the human subject 100 is addictive to the Internet.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method of estimating Internet activity dependence of a human subject, to be implemented by a system that includes a respiration sensing unit and a processing unit, said method comprising the steps of:
A) by the respiration sensing unit, sensing respiration-caused thorax and abdomen movements of the human subject during a first time period, and a subsequent second time period during which an emotional stimulation material played on a multimedia playing unit is being watched by the human subject so as to generate a first thoracic respiratory signal (St₁) and a first abdominal respiratory signal (Sa₁) that correspond to the first time period, and a second thoracic respiratory signal (St₂) and a second abdominal respiratory signal (Sa₂) that correspond to the second time period; and
B) by the processing unit, generating an estimation result associated with the Internet activity dependence of the human subject based on a criterion associated with the emotional stimulation material, the first and second thoracic respiratory signals (St₁, St₂), and the first and second abdominal respiratory signals (Sa₁, Sa₂).

2. The method as claimed in claim 1, wherein step B) includes the sub-steps of:
B-1) by the processing unit, dividing the first thoracic respiratory signal (St₁) into a first signal component (St₁₁) and a second signal component (St₁₂) whose frequencies differ from each other, the first abdominal respiratory signal (Sa₁) into a first signal component (Sa₁₁) and a second signal component (Sa₁₂) whose frequencies differ from each other, the second thoracic respiratory signal (St₂) into a first signal component (St₂₁) and a second signal component (St₂₂) whose frequencies differ from each other, and the second abdominal respiratory signal (Sa₂) into a first signal component (Sa₂₁) and a second signal component (Sa₂₂) whose frequencies differ from each other;
B-2) by the processing unit, calculating an average frequency (Ft₁₁) and an average amplitude (At₁₁) of the first signal component (St₁₁), an average frequency (Ft₁₂) and an average amplitude (At₁₂) of the second signal component (St₁₂), an average frequency (Fa₁₁) and an average amplitude (Aa₁₁) of the first signal component (Sa₁₁), an average frequency (Fa₁₂) and an average amplitude (Aa₁₂) of the second signal component (Sa₁₂), an average frequency (Ft₂₁) and an average amplitude (At₂₁) of the first signal component (St₂₁), an average frequency (Ft₂₂) and an average amplitude (At₂₂) of the second signal component (St₂₂), an average frequency (Fa₂₁) and an average amplitude (Aa₂₁) of the first signal component (Sa₂₁), and an average frequency (Fa₂₂) and an average amplitude (Aa₂₂) of the second signal component (Sa₂₂), the average frequencies (Ft₁₁, Fa₁₁, Ft₂₁, Fa₂₁) and the average amplitudes (At₁₁, Aa₁₁, At₂₁, Aa₂₁) cooperatively constituting first data, the average frequencies (Ft₁₂, Fa₁₂, Ft₂₂, Fa₂₂) and the average amplitudes (At₁₂, Aa₁₂, At₂₂, Aa₂₂) cooperatively constituting second data; and
B-3) by the processing unit, generating the estimation result based on at least one of the first data and the second data, and on the emotional stimulation material.

3. The method as claimed in claim 2, wherein step B) further includes, prior to step B-1), the sub-step of:
B-0) by the processing unit, filtering out noises, which have frequencies higher than 50 Hz or lower than 0.01 Hz, in the first and second thoracic respiratory signals (St₁, St₂), and in the first and second abdominal respiratory signals (Sa₁, Sa₂).

4. The method as claimed in claim 2, wherein, in sub-step B-1), the processing unit obtains the first signal components (St₁₁, Sa₁₁, St₂₁, Sa₂₁) and the second signal components (St₁₂, Sa₁₂, St₂₂, Sa₂₂) using a decomposition method associated with a complementary ensemble empirical model.

5. The method as claimed in claim 2, wherein:
in step A), the emotional stimulation material includes positive emotional content that tends to stimulate positive emotions in a viewer thereof; and
sub-step B-3) includes
by the processing unit, comparing the average frequency (Ft₁₁) with the average frequency (Ft₂₁), the average frequency (Fa₁₁) with the average frequency (Fa₂₁), the average amplitude (At₁₁) with the average amplitude (At₂₁), and the average amplitude (Aa₁₁) with the average amplitude (Aa₂₁) so as to generate a first comparison result, and
by the processing unit, generating the estimation result based on the criterion associated with the emotional stimulation material and on the first comparison result.

6. The method as claimed in claim 5, wherein:
when the first comparison result indicates that the average frequency (Ft₁₁) is greater than the average frequency (Ft₂₁), that the average frequency (Fa₁₁) is greater than the average frequency (Fa₂₁), that the average amplitude (At₁₁) is smaller than the average amplitude (At₂₁) and that the average amplitude (Aa₁₁) is smaller than the average amplitude (Aa₂₁), the processing unit generates a first estimation output indicating that the human subject is relatively highly dependent on Internet activity and serving as the estimation result; and
when the first comparison result indicates that the average frequency (Ft₁₁) is greater than the average frequency (Ft₂₁), that the average frequency (Fa₁₁) is smaller than the average frequency (Fa₂₁), that the average amplitude (At₁₁) is greater than the average amplitude (At₂₁) and that the average amplitude (Aa₁₁) is greater than the average amplitude (Aa₂₁), the processing unit generates a second estimation result indicating that the human subject is relatively lowly dependent on Internet activity and serving as the estimation result.

7. The method as claimed in claim 5, wherein:
sub-step B-3) further includes, prior to generation of the estimation result, by the processing unit, comparing the average frequency (Ft₁₂) with the average frequency (Ft₂₂), the average frequency (Fa₁₂) with the average frequency (Fa₂₂), the average amplitude (At₁₂) with the average amplitude (At₂₂), and the average amplitude (Aa₁₂) with the average amplitude (Aa₂₂) so as to generate a second comparison result; and
the processing unit generates the estimation result based on the criterion associated with the emotional stimulation material and the first comparison result and further on the second comparison result.

8. The method as claimed in claim 7, wherein:
when the first comparison result indicates that the average frequency (Ft₁₁) is greater than the average frequency (Ft₂₁), that the average frequency (Fa₁₁) is greater than the average frequency (Fa₂₁), that the average amplitude (At₁₁) is smaller than the average amplitude (At₂₁) and that the average amplitude (Aa₁₁) is smaller than the average amplitude (Aa₂₁), while the second comparison result indicates that the average frequency (Ft₁₂) is greater than the average frequency (Ft₂₂), that the average frequency (Fa₁₂) is smaller than the average frequency (Fa₂₂), that the average amplitude (At₁₂) is greater than the average amplitude (At₂₂) and that the average amplitude (Aa₁₂) is greater than the average amplitude (Aa₂₂), the processing unit generates, a first estimation output indicating that the human subject is relatively highly dependent on Internet activity and serving as the estimation result; and
when the first comparison result indicates that the average frequency (Ft₁₁) is greater than the average frequency (Ft₂₁), that the average frequency (Fa₁₁) is smaller than the average frequency (Fa₂₁), that the average amplitude (At₁₁) is greater than the average amplitude (At₂₁) and that the average amplitude (Aa₁₁) is greater than the average amplitude (Aa₂₁), while the second comparison result indicates that the average frequency (Ft₁₂) is smaller than the average frequency (Ft₂₂), that the average frequency (Fa₁₂) is smaller than the average frequency (Fa₂₂), that the average amplitude (At₁₂) is smaller than the average amplitude (At₂₂) and that the average amplitude (Aa₁₂) is smaller than the average amplitude (Aa₂₂), the processing unit generates, a second estimation output indicating that the human subject is relatively lowly dependent on Internet activity and serving as the estimation result.

9. The method as claimed in claim 2, wherein:
in step A), the emotional stimulation material includes positive emotional content that tends to stimulate positive emotions in a viewer thereof; and
sub-step B-3) includes
by the processing unit, comparing the average frequency (Ft₁₂) with the average frequency (Ft₂₂), the average frequency (Fa₁₂) with the average frequency (Fa₂₂), the average amplitude (At₁₂) with the average amplitude (At₂₂), and the average amplitude (Aa₁₂) is with the average amplitude (Aa₂₂) so as to generate a comparison result, and
by the processing unit, generating the estimation result based on the criterion associated with the emotional stimulation material and on the comparison result.

10. The method as claimed in claim 9, wherein:
when the comparison result indicates that the average frequency (Ft₁₂) is greater than the average frequency (Ft₂₂), that the average frequency (Fa₁₂) is smaller than the average frequency (Fa₂₂), that the average amplitude (At₁₂) is greater than the average amplitude (At₂₂) and that the average amplitude (Aa₁₂) is greater than the average amplitude (Aa₂₂), the processing unit generates a first estimation output indicating that the human subject is relatively highly dependent on Internet activity and serving as the estimation result; and
when the comparison result indicates that the average frequency (Ft₁₂) is smaller than the average frequency (Ft₂₂), that the average frequency (Fa₁₂) is smaller than the average frequency (Fa₂₂), that the average amplitude (At₁₂) is smaller than the average amplitude (At₂₂) and that the average amplitude (Aa₁₂) is smaller than the average amplitude (Aa₂₂), the processing unit generates a second estimation result indicating that the human subject is relatively lowly dependent on Internet activity and serving as the estimation result.

11. The method as claimed in claim 2, wherein:
in step A), the emotional stimulation material includes negative emotional content that tends to stimulate negative emotions in a viewer thereof; and
sub-step B-3) includes
by the processing unit, comparing the average frequency (Ft₁₁) with the average frequency (Ft₂₁), the average frequency (Fa₁₁) with the average frequency (Fa₂₁), the average amplitude (At₁₁) with the average amplitude (At₂₁), and the average amplitude (Aa₁₁) with the average amplitude (Aa₂₁) so as to generate a first comparison result, and
by the processing unit, generating the estimation result based on the criterion associated with the emotional stimulation material and on the first comparison result.

12. The method as claimed in claim 11, wherein:
when the first comparison result indicates that the average frequency (Ft₁₁) is greater than the average frequency (Ft₂₁), that the average frequency (Fa₁₁) is greater than the average frequency (Fa₂₁), that the average amplitude (At₁₁) is greater than the average amplitude (At₂₁) and that the average amplitude (Aa₁₁) is smaller than the average amplitude (Aa₂₁), the processing unit generates a first estimation output indicating that the human subject is relatively highly dependent on Internet activity and serving as the estimation result; and
when the first comparison result indicates that the average frequency (Ft₁₁) is smaller than the average frequency (Ft₂₁), that the average frequency (Fa₁₁) is smaller than the average frequency (Fa₂₁), that the average amplitude (At₁₁) is smaller than the average amplitude (At₂₁) and that the average amplitude (Aa₁₁) is greater than the average amplitude (Aa₂₁), the processing unit generates a second estimation result indicating that the human subject is relatively lowly dependent on Internet activity and serving as the estimation result.

13. The method as claimed in claim 11, wherein:
sub-step B-3) further includes, prior to generation of the estimation result, by the processing unit, comparing the average frequency (Ft₁₂) with the average frequency (Ft₂₂), the average frequency (Fa₁₂) with the average frequency (Fa₂₂), the average amplitude (At₁₂) with the average amplitude (At₂₂), and the average amplitude (Aa₁₂) with the average amplitude (Aa₂₂) so as to generate a second comparison result; and
the processing unit generates the estimation result based on the criterion associated with the emotional stimulation material and the first comparison result and further on the second comparison result.

14. The method as claimed in claim 13, wherein:
when the first comparison result indicates that the average frequency (Ft₁₁) is greater than the average frequency (Ft₂₁), that the average frequency (Fa₁₁) is greater than the average frequency (Fa₂₁), that the average amplitude (At₁₁) is greater than the average amplitude (At₂₁) and that the average amplitude (Aa₁₁) is smaller than the average amplitude (Aa₂₁) while the second comparison result indicates that the average frequency (Ft₁₂) is smaller than the average frequency (Ft₂₂), that the average frequency (Fa₁₂) is greater than the average frequency (Fa₂₂), that the average amplitude (At₁₂) is smaller than the average amplitude (At₂₂) and that the average amplitude (Aa₁₂) is greater than the average amplitude (Aa₂₂), the processing unit generates a first estimation output indicating that the human subject is relatively highly dependent on Internet activity and serving as the estimation result; and
when the first comparison result indicates that the average frequency (Ft₁₁) is smaller than the average frequency (Ft₂₁), that the average frequency (Fa₁₁) is smaller than the average frequency (Fa₂₁), that the average amplitude (At₁₁) is smaller than the average amplitude (At₂₁) and that the average amplitude (Aa₁₁) is greater than the average amplitude (Aa₂₁) while the second comparison result indicates that the average frequency (Ft₁₂) is smaller than the average frequency (Ft₂₂), that the average frequency (Fa₁₂) is greater than the average frequency (Fa₂₂), that the average amplitude (At₁₂) is greater than the average amplitude (At₂₂) and that the average amplitude (Aa₁₂) is smaller than the average amplitude (Aa₂₂), the processing unit generates a second estimation output indicating that the human subject is relatively lowly dependent on Internet activity and serving as the estimation result.

15. The method as claimed in claim 2, wherein:
in step A), the emotional stimulation material includes negative emotional content that tends to stimulate negative emotions in a viewer thereof; and
sub-step B-3) includes
by the processing unit, comparing the average frequency (Ft₁₂) with the average frequency (Ft₂₂), the average frequency (Fa₁₂) with the average frequency (Fa₂₂), the average amplitude (At₁₂) with the average amplitude (At₂₂), and the average amplitude (Aa₁₂) with the average amplitude (Aa₂₂) so as to generate a comparison result, and
by the processing unit, generating the estimation result based on the criterion associated with the emotional stimulation material and on the comparison result.

16. The method as claimed in claim 15, wherein:
when the comparison result indicates that that the average frequency (Ft₁₂) is smaller than the average frequency (Ft₂₂), that the average frequency (Fa₁₂) is greater than the average frequency (Fa₂₂), that the average amplitude (At₁₂) is smaller than the average amplitude (At₂₂) and that the average amplitude (Aa₁₂) is greater than the average amplitude (Aa₂₂), the processing unit generates a first estimation output indicating that the human subject is relatively highly dependent on Internet activity and serving as the estimation result; and
when the comparison result indicates that that the average frequency (Ft₁₂) is smaller than the average frequency (Ft₂₂), that the average frequency (Fa₁₂) is greater than the average frequency (Fa₂₂), that the average amplitude (At₁₂) is greater than the average amplitude (At₂₂) and that the average amplitude (Aa₁₂) is smaller than the average amplitude (Aa₂₂), the processing unit generates a second estimation result indicating the human subject is relatively lowly dependent on Internet activity and serving as the estimation result.
